(19) United States
(12) Patent Application Publication
(10) Pub. No.: US 2023/0140560 A1
(43) Pub. Date: May 4, 2023

(54) ELECTRODE WITH PROTECTED EDGE REGION

(71) Applicant: Heraeus Deutschland GmbH & Co. KG, Hanau (DE)

(72) Inventors: Andreas LIESS, Hanau (DE); Leonard STOICA, Hanau (DE)

(21) Appl. No.: 18/051,724

(22) Filed: Nov. 1, 2022

(30) Foreign Application Priority Data
Nov. 2, 2021 (DE) 102021128427.9

(51) Int. Cl. H01B 5/14; H01B 1/12; A61B 5/268

(52) U.S. Cl. CPC H01B 5/14; H01B 1/127; A61B 5/268; A61B 2562/125

(57) ABSTRACT

The present invention relates to a medical electrode comprising a base body on which an electrically conductive first layer and a cover layer are arranged, wherein the cover layer comprises an overhang which partially covers the first layer.

### CROSS-REFERENCE TO RELATED APPLICATIONS

This application claims priority pursuant to 35 U.S.C. 119(a) to German Application No. 102021128427.9, filed Nov. 2, 2021, which application is incorporated herein by reference in its entirety.

### FIELD OF THE INVENTION

The present invention relates to the field of medical technology, in particular medical electrodes, which can be used, for example, for electrical stimulation, detection, or ablation in or on the human body.

### TECHNICAL BACKGROUND

Medical electrodes need to meet stringent requirements. The conductive elements of an electrode are frequently provided with a soft coating which is exposed to mechanical loads during the intended use and can thereby be damaged or even removed. Good protection against damage is desirable, particularly in the case of medical electrodes having a flexible base body. Examples of medical electrodes and their production are disclosed in US2019290898A1 and EP3284509A1.

### PREFERRED EMBODIMENTS

The object of the present invention is to solve one or more of the problems illustrated above and further problems of the prior art. For example, the invention enables the production of particularly stable medical electrodes which are well protected against damage. Furthermore, the present invention provides electrodes with improved stability, performance, handleability, and/or measuring sensitivity.

These objects are achieved by the methods and apparatuses described herein, in particular those that are described in the claims.

Preferred embodiments of the invention are described below.
1. A medical electrode comprising a base body on which an electrically conductive first layer and a cover layer are arranged, wherein the cover layer comprises an overhang which partially covers the first layer.
2. The medical electrode according to embodiment 1, wherein the cover layer comprises a recess.
3. The medical electrode according to embodiment 2, wherein the recess tapers or widens toward the base body.
4. The medical electrode according to any one of the preceding embodiments, wherein the overhang comprises a side wall arranged at an angle (A) to the surface of the first layer that is (i) less than 80° or (ii) 110° to 170°.
5. The medical electrode according to any one of the preceding embodiments, further comprising an electrically conductive second layer arranged on the first layer.
6. The medical electrode according to embodiment 5, wherein the second layer comprises an electrically conductive polymer.
7. The medical electrode according to embodiment 6, wherein the electrically conductive polymer comprises PEDOT.
8. The medical electrode according to any one of the preceding embodiments 5 to 7, wherein the second layer is arranged exclusively within the recess, wherein the outward-facing surface of the second layer is spaced apart from the outward-facing surface of the cover layer.
9. The medical electrode according to any one of the preceding embodiments, wherein the base body comprises a flexible polymer substrate.
10. The medical electrode according to any one of the preceding embodiments, further comprising a conductor track which is electrically conductively connected to the first layer.
11. The medical electrode according to any one of the preceding embodiments, wherein the overhang is arranged and configured to mechanically stabilize the first layer.
12. The medical electrode according to any one of the preceding embodiments, wherein the overhang is arranged and configured to enable an electrical charge exchange between a liquid external medium and the first or optionally second layer from different directions.
13. The medical electrode according to any one of the preceding embodiments, wherein the overhang is arranged and configured to protect the base body against the penetration of liquid.
14. The medical electrode according to any one of the preceding embodiments, wherein the edge of the first layer is substantially completely covered by the cover layer.
15. A use of a medical electrode according to any one of the preceding embodiments in a device which is configured for electrical stimulation, detection, or ablation.
16. A method for producing a medical electrode, comprising the steps of:
   (i) providing a base body on which an electrically conductive first layer is arranged,
   (ii) arranging a cover layer on the base body so that the first layer is partially covered by an overhang of the cover layer.
17. The method according to embodiment 16, wherein the overhang comprises a side wall arranged at an angle (A) to the surface of the first layer that is (i) less than 80° or (ii) 110° to 170°.
18. An electrode system comprising a plurality of electrodes according to any one of embodiments 1 to 14.
19. A method for medical treatment, comprising contacting an electrode according to any one of embodiments 1 to 14 or an electrode system according to embodiment 18 with a subject.
20. A method for medical diagnosis, comprising contacting an electrode according to any one of embodiments 1 to 14 or an electrode system according to embodiment 18 with a subject.

### DETAILED DESCRIPTION

In principle, for the embodiments described herein, the elements of which “contain” or “comprise” a particular feature (e.g., a material), a further embodiment is always considered in which the element in question consists of that feature alone, i.e., comprises no further components. The word “comprise” or “comprising” is used herein synonymously with the word “contain” or “containing”.

If an element is referred to in the singular in an embodiment, an embodiment is also being considered in which several of these elements are present. The use of a term for an element in the plural fundamentally also encompasses an embodiment in which only a single corresponding element is contained.

Unless otherwise indicated or clearly precluded from the context, it is possible in principle, and is herewith clearly taken into consideration, that features of different embodiments may also be present in the other embodiments described herein. It is also contemplated in principle that all features that are described herein in conjunction with a method are also applicable to the products and apparatuses described herein, and vice versa. Only for reasons of succinct presentation are all such contemplated combinations not explicitly listed in all instances. Technical solutions which are known to be equivalent to the features described herein are also intended to be encompassed in principle by the scope of the invention.

A first aspect of the invention relates to a medical electrode comprising a base body on which an electrically conductive first layer and a cover layer are arranged, wherein the cover layer comprises an overhang which partially covers the first layer.

A medical electrode within the meaning of the invention is intended for use on a subject, i.e., on or in the human or animal body. When such an electrode is used as intended, an electrical signal is sent to the body and/or received from the body. In a preferred embodiment, a medical electrode is therefore configured to send an electrical signal to the body and/or to receive an electrical signal from the body. For example, nerve or muscle tissue can be functionally stimulated by means of an electrical signal, or such a tissue can output electrical signals which are received by a medical electrode according to the invention. “Functionally stimulate” herein means the stimulation of a physiological response, for example the contraction of a muscle, or an action potential of a neuron. Another example is the removal (ablation) of tissue using electrical impulses that can be sent to the tissue by a medical electrode according to the invention.

The medical electrode according to the invention comprises a base body. The base body preferably comprises an electrically insulating material which is configured to support an electrically conductive layer.

The base body preferably comprises a polymer. In one embodiment, the base body comprises a flexible polymer substrate. “Flexible polymer substrate” is understood to mean a polymer body that can be deformed with bare hands without any particular effort. The flexible polymer substrate is preferably a polymer film. Examples of materials that can be used in connection with the base body according to the invention include polyester, polyethylene foam, cellulose nonwoven, polyethylene vinyl acetate, polyurethane, epoxy resins, liquid-crystalline polymers and polyimide, or mixtures or material composites thereof.

Examples of liquid-crystalline polymers include LCP polyimide, 1CP-BT epoxy, and mixtures thereof. They are inter alia commercially available from Dyconex (Bassersdorf, Switzerland).

The base body may also comprise multilayer systems, for example of different polymers. The base body may comprise polyimide, for example, and be coated with an epoxy resin. Examples of suitable polymers that may be used for the base body also include commercially available solder resist paints, which are obtainable from Dyconex, for example.

The electrode comprises an electrically conductive first layer. Said electrically conductive first layer is preferably configured to send or to receive an electrical signal. The terms “electrically conductive” and “electrically conducting” are used synonymously here and denote an electrical conductivity of a material, as is usual and expedient in connection with a medical electrode.

The electrically conductive first layer preferably comprises a metal or an alloy. Examples of usable metals are gold, platinum, nickel, palladium, iridium, titanium, silver, copper, and iron. Examples of usable alloys are stainless steel, MP35, or a platinum-iridium alloy.

In some embodiments, the electrically conductive first layer comprises an alloy, such as MP35, PtIr10, PtIr20, 316L, 301, or nitinol.

In one embodiment, the first layer comprises a metal or an alloy selected from the group consisting of gold, platinum, stainless steel, nitinol, and a platinum-iridium alloy. Examples of stainless steel include 316L and 301. Examples of platinum-iridium alloys include PtIr10 and PtIr20.

MP35 is a nickel-cobalt-based hardenable alloy. A variant of MP35 is described in industry standard ASTM F562-13. In one embodiment, MP35 is an alloy comprising 33 to 37% Co, 19 to 21% Cr, 9 to 11% Mo, and 33 to 37% Ni.

PtIr10 is an alloy of 88 to 92% platinum and 8 to 12% iridium.

PtIr20 is an alloy of 78 to 82% platinum and 18 to 22% iridium.

316L is an acid-resistant CrNiMo austenitic steel with approx. 17% Cr; approx. 12% Ni, and at least 2.0% Mo. A variant of 316L is described in industry standard 10088-2. In one embodiment, 316L is an alloy comprising 16.5 to 18.5% Cr; 2 to 2.5% Mo, and 10 to 13% Ni.

301 is a chromium-nickel steel with high corrosion resistance. A variant of 301 is described in industry standard DIN 1.4310. In one embodiment, 301 is an alloy comprising 16 to 18% Cr and 6 to 8% Ni.

Nitinol is a shape-memory nickel-titanium alloy having an ordered cubic crystal structure and a nickel content of approximately 55%, the remaining portion consisting of titanium. Nitinol has good biocompatibility and corrosion resistance properties.

Unless otherwise indicated, all percentages given herein are to be understood as weight percent (wt. %).

The first layer can have a fine structure on its surface, as shown for example in EP3108927A1, which is hereby fully incorporated by reference. In one embodiment, the first layer comprises: microprotrusions, macroprotrusions, wherein the microprotrusions are arranged on the macroprotrusions, a first set of recesses, wherein the first set of recesses comprises at least two longitudinal recesses, wherein the macroprotrusions and the at least two longitudinal recesses are arranged in an alternating pattern, wherein at least 50% of the macroprotrusions have a width, measured along a first direction, in the range of 2.0 μm to 40.0 μm; at least 50% of the microprotrusions have a width, measured along the first direction, in the range from 0.001 μm to 1.000 μm.

A cover layer is arranged on the base body. Said cover layer comprises an overhang covering the electrically conductive first layer partially but not completely. The cover layer preferably consists of an electrically non-conductive material, such as a polymer. Examples of suitable polymers include compounds selected from the group consisting of a liquid-crystalline polymer, medical silicone, an epoxy resin, polyurethane, polyethylene, polyacrylic, P(M)MA, ABS, PVDF, polyester, polyamide, polyimide, SEBS, PEEK, PPS, PEVA, PEN, polysulfones, and copolymers and mixtures thereof.

The base body and the cover layer can consist of the same material or of different materials in each case.

In one embodiment, the cover layer covers, in each case at least partially, both the base body and the first layer.

The cover layer preferably covers the edge of the electrically conductive first layer, wherein “edge” means the outer edge of the first layer, which, in a cross-sectional view, is arranged distally to the base body, i.e., faces in the direction opposite to the base body and has a maximum distance from the center point of the first layer.

In some embodiments, the edge of the first layer is substantially completely covered by the cover layer (as shown in each case in the figures).

In a preferred embodiment, the cover layer completely covers the edge of the electrically conductive first layer.

The above-described overlapping arrangement of the cover layer with respect to the electrically conductive first layer provides a number of technical advantages. The electrically conductive first layer is thereby stabilized. For example, the overhang protects the first layer against delamination, i.e., against removal from the base body. In addition, the overhang protects against diffusion between the first layer and the base body when the first layer is coated in a liquid coating process, as is described in more detail elsewhere herein. Overall, during the production and use of the electrode, the deeper layers are protected against external influences, for example against oxidation.

The cover layer can furthermore comprise a recess. The recess can enable access to the electrically conductive first layer. The recess can be delimited by side walls of the overhang. The recess can have different geometries. For example, the recess can taper in a funnel shape toward the base body, or it can widen toward the base body. This can be conditional upon different angles of the side walls of the overhang with respect to the surface of the first layer.

In principle, the side walls can be arranged at any angle. For example, the overhang can be arranged at an angle of 90° or approximately 90°, for example 85° to 95°, to the surface of the first layer However, it may be advantageous if this angle deviates significantly from 90°.

For example, the overhang can be arranged at an angle of less than 80° to the surface of the first layer so that the recess widens toward the base body and forms a cavity or hollow. Alternatively, this angle can be, for example, 110° to 170° so that the recess tapers toward the base body so that the recess has a funnel shape. Said angle is defined as shown in the accompanying drawings (referred to there as angle “A”), i.e., it is measured between a side wall of the overhang and the surface of the first layer on the “inner side”, i.e., the side of the recess.

These two variants can bring about various advantages. An angle of more than 90° (“funnel shape”), for example approximately 135°, can protect the cover layer against mechanical damage since there are fewer exposed protruding edges on the outward-facing side of the cover layer in comparison to orthogonally steep side walls. Therefore, smaller shear forces occur at the surface than in the case of an orthogonal shape. The protection against delamination of the cover layer and against penetrating impurities can also be improved with this “funnel shape”. In addition, with an identically sized accessible electrode area of the first layer, or the conductive coating thereof, a better charge exchange with the external medium can take place via diffusion, as is explained in more detail herein with reference to the drawings. With reference to the charge exchange of the electrode with the external medium, “diffusion” here means the movement of the charge carriers in an external medium, which movement can be caused, for example, by applying an electrical voltage. Accordingly, the term “diffusion” is not limited herein to Brownian motion in this context but instead generally describes the movement of charge carriers, for example ions, in a liquid medium, for bringing about a charge exchange or electrical current flow.

As a result of the “funnel shape”, the electrode can also be better contacted with a tissue of a subject.

The external medium can, for example, be a tissue fluid of a subject. In order to examine the charge exchange with the external medium of the electrode, it is also possible, for example, to carry out an in-vitro measurement in physiological saline solution or another salt solution, such as a KCl solution, as is customary in the art.

When the angle of the side walls is less than 90°, the overhang forms a partially delimited region above the first layer, which acts as a “dead volume”. This results in the first layer being more strongly shielded from the external medium. This may be advantageous for rendering the electrode more independent against interference signals or for “capturing” diffusing particles within the recess, and better detecting them thereby in certain cases.

In some embodiments, the medical electrode furthermore comprises an electrically conductive second layer arranged on the first layer. The first layer may be coated, for example, with a conductive polymer or a metal, alloy, or metal oxide. Suitable metals are, for example, platinum, gold, or other medically compatible precious metals and medically compatible alloys. An example of a suitable metal oxide is iridium oxide.

The conductive polymer may comprise, for example, a polymer selected from the group consisting of a polyacetylene, a polyvinyl alcohol, a polyfluorene, a polyphenylene, a polyphenylene vinylene, a polypyrene, a polyazulene, a polynaphthalene, a polypyrrole, a polycarbazole, a polyindole, a polyazepine, a polyaniline, a polyacene, a polythiophene, a polythiophene vinylene, a polyphenylene sulfide, a polypyridine or functionalized derivatives, precursors or mixtures thereof. Examples of conductive polymers are described in WO 2015/031265 A1, which is hereby incorporated fully by reference.

In some embodiments, the conductive polymer comprises poly-3,4-ethylenedioxythiophene (PEDOT). In one embodiment, the conductive polymer comprises PEDOT:PSS, i.e., PEDOT that is complexed with polystyrene sulfonate. In one embodiment, the conductive polymer comprises PEDOT:PSS and a further polymer. One example of such a further polymer is PVP (polyvinylpyrrolidone).

Suitable conductive polymers are commercially available, for example the CLEVIOS® and AMPLICOAT® products from Heraeus (Hanau, Germany). In particular, the AMPLICOAT® product is advantageous for use in implantable medical devices. The designation AMPLICOAT herein relates to both the commercially available precursor substance and the polymer produced therefrom.

Preferably, the first layer and the second layer comprise different materials. For example, the first layer may comprise platinum or a platinum-iridium alloy, and the second layer may comprise PEDOT, or a PEDOT-containing composition, in particular AMPLICOAT®.

In a further embodiment, the first layer may comprise platinum or a platinum-iridium alloy, and the second layer may comprise an iridium oxide layer.

In one embodiment, the first layer comprises gold, and the second layer comprises PEDOT.

In one embodiment, the first layer comprises platinum, and the second layer comprises PEDOT.

In one embodiment, the first layer comprises a platinum-iridium alloy, and the second layer comprises PEDOT. In this case, the second layer can in each case comprise, for example, a PEDOT-containing composition, in particular AMPLICOAT®, as described in more detail elsewhere herein. In one embodiment, the first layer comprises gold and the second layer comprises AMPLICOAT.

In one embodiment, the first layer comprises gold and the second layer comprises iridium oxide.

In one embodiment, the first layer comprises platinum and the second layer comprises iridium oxide.

In one embodiment, the first layer comprises a platinum-iridium alloy, and the second layer comprises iridium oxide. In some embodiments, a layer comprising iridium oxide may be produced in each case by means of a thermally decomposable iridium-containing composition, as described in more detail elsewhere herein.

In one embodiment, the first layer comprises gold, and the second layer comprises platinum.

In one embodiment, the first layer comprises platinum and the second layer comprises platinum. In this case, the two platinum layers may, for example, have a different structure, such as a different surface quality and/or porosity.

In one embodiment, the first layer comprises a platinum-iridium alloy, and the second layer comprises platinum. The second layer of platinum can in each case be produced by means of a thermally decomposable platinum-containing composition, as described in more detail elsewhere herein.

Liquid coating methods can be used to produce the second layer. AMPLICOAT can preferably be applied by electrodeposition.

The conductive polymers described herein can also be provided with dopants. Doping can increase the conductivity of a polymer and create a lower energy threshold for the conductivity. Dopants can also contribute to controlling the conductivity properties in a targeted manner There are many methods and materials which are useful for doping and which should be known to the person skilled in the art. The dopants include, inter alia, chloride, polystyrene sulfonate (PSS), dodecylbenzene sulfonate, naphthalene sulfonate, and lithium perchlorate.

The electrically conductive second layer is preferably deposited by an electrodeposition process (also referred to as “electropolymerization”), as is known to the person skilled in the art.

“Electrodeposition” is understood to mean the deposition of a material by applying an electric potential between two conductive materials (or electrodes) in a liquid medium containing charged substances. In various embodiments, the materials are galvanically deposited on the anode (i.e., on the electrode on which the monomer oxidation takes place). Typical apparatuses for carrying out the electrodeposition include the following: an anode, a cathode, and frequently a reference electrode, each separated by an electrolyte (e.g., an ion-containing solution), and a potentiostat which monitors/adjusts the voltages/currents on the different electrodes. The electrochemical deposition can be carried out under various electrochemical conditions, inter alia under the following conditions: (a) constant current, (b) constant voltage, (c) current scan/sweep, e.g., via single or multiple scans/sweeps, (d) voltage scan/sweep, e.g., via single or multiple scans/sweeps, (e) current square waves or other current pulse waveforms, (f) voltage square waves or other voltage pulse waveforms, and (g) a combination of different current and voltage parameters.

The process of galvanic deposition may be controlled such that layers of conductive polymer of different thicknesses are deposited.

The second layer may completely or partially cover the exposed part of the first layer. In this context, the “exposed part of the first layer” means the part of the first layer that is not covered directly by the cover layer. The second layer can be applied in a geometric shape that differs from the shape of the surface of the first layer. For example, the exposed part of the first layer may have a square-shaped surface, and the surface of the second layer may be circular in shape.

In some embodiments, the second layer is arranged exclusively within the recess of the cover layer. This means that the second layer does not protrude out of the recess, and in particular does not cover the surface of the cover layer.

Furthermore, it may be advantageous for the outward-facing surface of the second layer to be spaced apart from the outward-facing surface of the cover layer. This means that although the second layer covers, for example completely covers, the exposed part of the first layer, it does not completely fill the recess. Rather, a free region remains within the recess, below the outward-facing surface of the cover layer. As a result, the second layer is better protected against mechanical damage. This is in particular advantageous if the second layer is mechanically sensitive, for example if the second layer comprises an electrically conductive polymer.

The present invention is well suited for use in flexible base bodies. In some embodiments, the base body therefore comprises a flexible polymer substrate. Examples of such flexible polymer substrates are familiar to the person skilled in the art under the term “Flex PCB” and are described in more detail elsewhere herein.

This makes it possible to produce flexible electrodes which present a lower risk of injury to patients and can be positioned better in the body than electrodes having a rigid structure.

In some embodiments, the medical electrode furthermore comprises a conductor track which is electrically conductively connected to the first layer. This conductor track serves to electrically contact the first layer. For example, an electrical signal can be sent to the first layer via the conductor track so that the signal can subsequently be sent from the first layer to a subject in order to bring about an electrical stimulation of the subject. The conductor track can be electrically conductively connected to the first layer directly or via further elements. For example, the conductor track can comprise copper and can be electrically conductively connected to the first layer via a barrier layer comprising nickel.

In one embodiment, the overhang is arranged and configured to mechanically stabilize the first layer. This can be achieved, for example, by appropriate selection of the angle of the side walls with respect to the first layer or second layer, as described in more detail elsewhere herein. As a result, the edge regions of the first layer, for example, can be protected against mechanical action.

In one embodiment, the overhang is arranged and configured to enable an electrical charge exchange between a liquid external medium and the layer from different directions by means of diffusion. For example, this may be achieved by appropriate selection of the angle of the side walls with respect to the first layer, as described herein. This can be advantageous for detection applications in particular since, in some cases, the sensitivity of measurements can be increased as a result.

In one embodiment, the overhang is arranged and configured to protect the base body against the penetration of liquid. For example, this may be achieved by appropriate selection of the angle of the side walls with respect to the first layer or second layer, as described herein.

The first layer and/or second layer may also be modified with further reagents, such as enzymes or antibodies. As a result, non-electrogenic analytes, such as proteins or uncharged organic substances, can be detected by means of the electrode by coupling them to an electrochemical reaction. Such methods are commonly used for determining blood glucose, for example. In particular, the outermost layer of the first or second layer can be modified in this case.

A further aspect of the invention relates to an electrode system which comprises a plurality of electrodes described herein. The system preferably comprises a plurality of the electrodes described herein, which can be electrically addressed independently of one another. As a result, for example, various sites of a target tissue of a subject can be electrically stimulated or detected independently of one another.

A further aspect of the invention relates to the use of a medical electrode described herein in a device which is configured for electrical stimulation, detection, or ablation. For example, the device may be a catheter for electrophysiological stimulation or tissue ablation. Further possible applications are, for example, cardiac pacemakers, implantable cardioverters, defibrillation devices and heart resynchronization devices, as well as implantable electrodes for neuromodulation, cardiac stimulation, deep brain stimulation, spinal cord stimulation, or gastric stimulation. In addition, the electrodes described herein can be used for ECG or EEG measurements, i.e., for examining heart or brain function. Moreover, the electrodes described herein can be used for detecting certain substances, for example proteins or metabolites. In some embodiments, the application of an electrode described herein includes both diagnostic and therapeutic functions. For example, the effect of a therapeutic function can be monitored, controlled, or checked, simultaneously or with a time delay, using a diagnostic function.

The present invention also relates to methods for medical treatment and methods for medical diagnosis in which the medical electrodes described herein are contacted with a patient. Such a method may include, for example, the application of an electrode described herein for neuromodulation, cardiac stimulation, deep brain stimulation, spinal cord stimulation, or gastric stimulation. Examples of medical diagnostic methods include ECG or EEG measurements.

A further aspect of the invention relates to a method for producing a medical electrode, comprising the steps of: providing a base body on which an electrically conductive first layer is arranged, arranging a cover layer on the base body so that the first layer is partially covered by an overhang of the cover layer.

In one embodiment, the method comprises structuring the cover layer using laser ablation. Alternatively, commonly used mechanical methods can also be used for processing the geometry of the cover layer, or the cover layer can be produced directly by means of a shaping method, for example injection molding, thermoplastic deformation, or 3D printing methods.

The method can furthermore provide suitable steps for arranging the individual components of the electrode in order to produce the embodiments described herein of the electrode. The features of all embodiments described herein of the electrode are therefore also correspondingly applicable in the method described herein.

For example, in the method according to the invention, the cover layer can be arranged on the base body so that the overhang comprises a side wall arranged at an angle to the surface of the first layer that is (i) less than 80° or (ii) 110° to 170°.

### EXAMPLES

The invention is further illustrated below using examples which are, however, not to be understood as limiting. It will be apparent to the person skilled in the art that other equivalent means may be similarly used in place of the features described here.

### Example 1—Wipe Test

Medical electrodes comprising a first layer of gold and a second layer of Amplicoat (layer thickness 1 micrometer) were produced. The first type of electrode A comprised a base body made of gold, embedded in PEEK, and had no cover layer. In contrast, the second type of electrode B comprised a base body made of polyimide, a first layer of gold (layer thickness 5 μm), and a cover layer which was arranged on the first layer and covered the edge of the first layer. Using a commercially available foam-tipped rod, the surfaces of the electrodes were wiped with a defined load of up to 203 g, i.e., the rod was guided laterally over the surfaces of the electrodes with a defined force. While with the first type of electrode A, delamination of the gold layer was observed starting from a load of approximately 118 g, no delamination was discernible in the second type of electrode B, even with a load of 203 g.

### FIGURES

FIG. 1 shows by way of example a cross-sectional view of an electrode according to a first embodiment of the invention. A first electrically conductive layer 102 is arranged on a base body 101. The first layer 102 is electrically contacted by means of a conductor track 104, which runs within the base body 101. The base body 101 and the first layer 102 are partially covered by a cover layer 103. The cover layer 103 comprises a recess 108 which is formed by the side walls of the cover layer 103 and by the first layer 102. The cover layer 103 comprises a protrusion 107 which partially overhangs and covers the surface of the first layer 102. This protrusion (overhang) protects the outer edge region of the first layer 102 and the underlying layers against mechanical damage and penetration of the external medium. As a result, the electrode is better protected against corrosion and delamination of the first layer 102. In this embodiment, the protrusion 107 has a rectangular geometry. The exposed surface of the first layer 102 may, for example, have a circular or rectangular geometry. Accordingly, the electrode may comprise one or more protrusions 107. In the event that the electrode comprises a plurality of protrusions 107, they are preferably identical in shape. In this example, the first electrically conductive layer 102 consists of PtIr10. The edge 109 of the first layer is completely covered by the protrusions 107 of the cover layer 103.

FIG. 2 shows by way of example a cross-sectional view of an electrode according to a second embodiment of the invention. The structure of the electrode is similar to that shown in FIG. 1, but the protrusion 107 comprises an oblique side wall 106. The side wall 106 is arranged at an angle A to the surface of the cover layer 103 that deviates significantly from 90°. This angle A is always defined here such that it is measured on the “inner side” of the cover layer, i.e., not toward the closest edge of the cover layer. An obtuse angle A (i.e., A greater than 90°) therefore means that the horizontal cross section of the recess 108 tapers downward (i.e., toward the first layer, away from the outer side of the cover layer). As a result, in comparison to the embodiment shown in FIG. 1, the outward-facing orthogonal edges of the cover layer 103, which are susceptible to damage, are dispensed with. If the side wall 106 is subjected to a force, for example, which acts parallel to the base body, the transfer of force to the interface of the cover layer 103 and the first layer 102 is reduced, which leads to greater mechanical stability of the cover layer 103. In addition, the recess 108 is given a funnel-shaped geometry.

FIG. 3 shows the charge exchange of the electrode according to FIG. 1 with the external medium. Due to the orthogonal geometry of the protrusion 107, the movement of the charge carriers in the external medium, which is shown by arrows in FIG. 3, runs in a parallel flow direction, perpendicularly to the surface of the first layer 102.

FIG. 4 shows the charge exchange of the electrode according to FIG. 2 with the external medium. Due to the funnel-shaped geometry of the recess 108, the movement of the charge carriers in the external medium, which is shown by arrows in FIG. 4, runs in a hemispherical flow direction, i.e., the charge exchange with the external medium can take place more effectively due to the oblique side wall 106 since the electrode has a larger “intake region”.

FIG. 5 shows by way of example a cross-sectional view of an electrode according to a third embodiment of the invention. In contrast to the embodiments shown in FIGS. 1 and 2, the angle A between the protrusion 106 and the surface of the first layer 102 is less than 90°, i.e., the cover layer 103 comprises an undercut in the region of the surface of the first layer 102. As a result, the edge regions of the first layer 102 are shielded more strongly from the external medium. As a result, a longer dwell time of analytes in the vicinity of the electrode surface can be achieved, for example. In this embodiment, the cover layer 103 also covers an edge region of the first layer 102, thereby protecting the first layer 102 against damage.

FIG. 6 shows by way of example a cross-sectional view of an electrode according to a further embodiment of the invention. In this case, a second layer 105 is arranged on the first layer 102. In this example, the first layer 102 consists of gold. In this example, the second layer 105 consists of Amplicoat, a conductive polymer comprising PEDOT. The second layer 105 is arranged exclusively within the recess of the cover layer, i.e., it does not extend over the outward-facing surface of the cover layer 103. In this example, the second layer 105 does not completely fill the recess: Rather, the outward-facing surface of the second layer 105 is spaced apart from the outward-facing surface of the cover layer 103 so that a free space remains in the recess 108. In this way, i.e., due to the recessed arrangement of the second layer 105 within the recess 108, the second layer 105 is protected against mechanical damage.

FIG. 7 shows by way of example a system having several electrodes according to the invention, which is shown in top view. Such a system may comprise any of the embodiments described herein. A plurality of electrodes is arranged at a uniform distance as a field. In this example, the angle A between the protrusion 106 and the surface of the first layer 102 is more than 90°, i.e., the recess 108 has a funnel shape, as also shown in FIG. 2. The side wall 106 is shown hatched. The protrusion 106 completely covers the edge of the first layer 102 but leaves the central region of the first layer 102 free so that it remains accessible to the external medium. Each of the six electrodes is electrically contacted by means of a conductor track (not shown in FIG. 7) and can be addressed independently of the other electrodes.

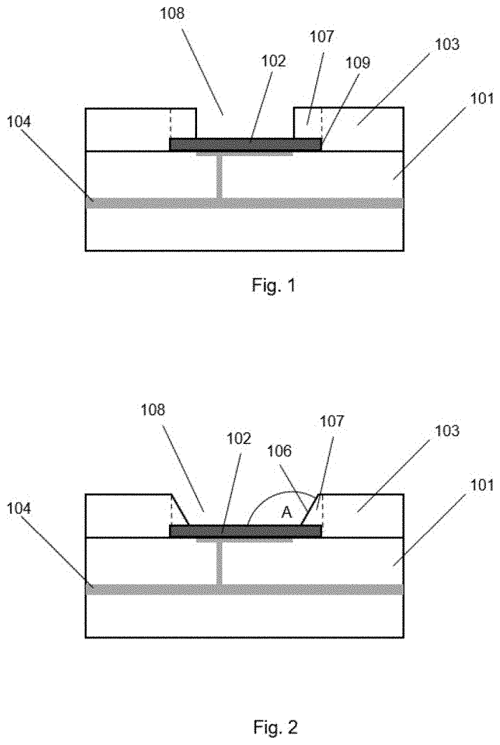

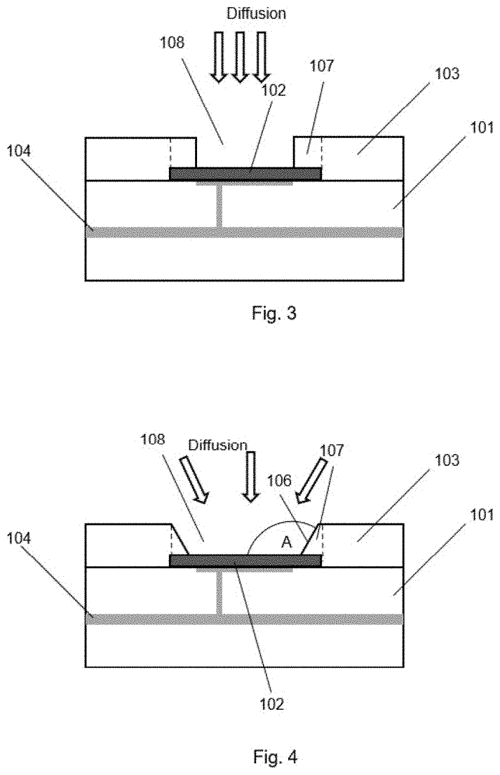

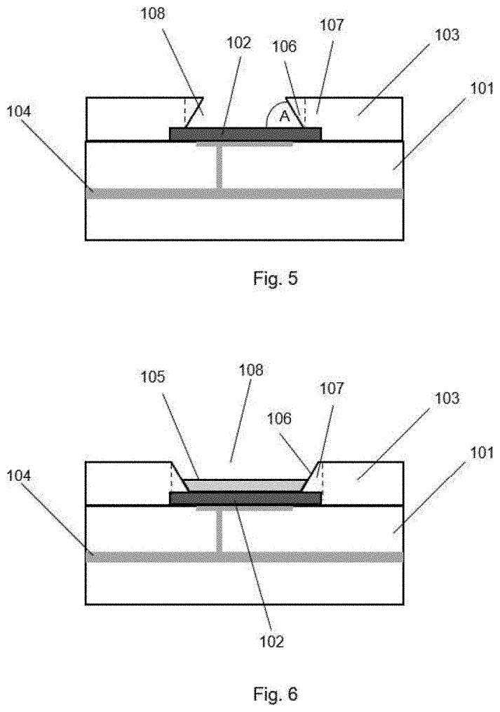

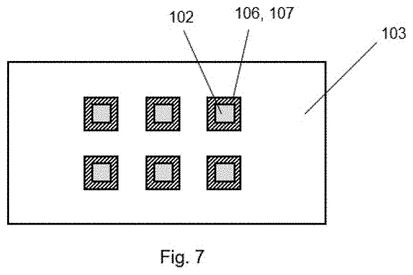

### LIST OF REFERENCE SIGNS

101 Base body
102 First layer
103 Cover layer
104 Conductor track
105 Second layer
106 Side wall
107 Protrusion
108 Recess
109 Edge of the first layer

What is claimed is:

1. A medical electrode, comprising a base body on which an electrically conductive first layer and a cover layer are arranged, wherein the cover layer comprises an overhang which partially covers the first layer.

2. The medical electrode according to claim 1, wherein the cover layer comprises a recess.

3. The medical electrode according to claim 2, wherein the recess tapers or widens toward the base body.

4. The medical electrode according to claim 1, wherein the overhang comprises a side wall arranged at an angle to the surface of the first layer that is (i) less than 80° or (ii) 110° to 170°.

5. The medical electrode according to claim 1, further comprising an electrically conductive second layer arranged on the first layer.

6. The medical electrode according to claim 5, wherein the second layer comprises an electrically conductive polymer.

7. The medical electrode according to claim 6, wherein the electrically conductive polymer comprises PEDOT.

8. The medical electrode according to claim 5, wherein the cover layer comprises a recess, wherein the second layer is arranged exclusively within the recess, wherein the outward-facing surface of the second layer is spaced apart from the outward-facing surface of the cover layer.

9. The medical electrode according to claim 1, wherein the base body comprises a flexible polymer substrate.

10. The medical electrode according to claim 1, further comprising a conductor track which is electrically conductively connected to the first layer.

11. The medical electrode according to claim 1, wherein the overhang is arranged and configured to mechanically stabilize the first layer.

12. The medical electrode according to claim 1, wherein the overhang is arranged and configured to enable an electrical charge exchange between a liquid external medium and the first layer and/or optionally the second layer from different directions.

13. The medical electrode according to claim 1, wherein the overhang is arranged and configured to protect the base body against the penetration of liquid.

14. The medical electrode according to claim 1, wherein the edge of the first layer is substantially completely covered by the cover layer.

15. A use of a medical electrode according to claim 1 in a device which is configured for electrical stimulation, detection, or ablation.

16. A method for producing a medical electrode, comprising the steps of:
(i) providing a base body on which an electrically conductive first layer is arranged, (ii) arranging a cover layer on the base body so that the first layer is partially covered by an overhang (107) of the cover layer.

17. The method according to claim 16, wherein the overhang comprises a side wall arranged at an angle to the surface of the first layer (102) that is (i) less than 80° or (ii) 110° to 170°.
